(19) United States
(12) Patent Application Publication
(10) Pub. No.: US 2003/0100002 A1
(43) Pub. Date: May 29, 2003

(54) Methods for preventing or reducing multiple antibiotic resistance in bacteria

(76) Inventor: Ambrose Cheung, Hanover, NH (US)

Correspondence Address: PENNIE AND EDMONDS, 1155 AVENUE OF THE AMERICAS, NEW YORK, NY, 100362711

(21) Appl. No.: 10/290,142

(22) Filed: Nov. 6, 2002

Related U.S. Application Data
(63) Continuation-in-part of application No. 10/092,264, filed on Mar. 6, 2002.
(60) Provisional application No. 60/273,791, filed on Mar. 6, 2001.
(60) Provisional application No. 60/312,546, filed on Aug. 15, 2001.
(60) Provisional application No. 60/329,140, filed on Oct. 12, 2001.

(51) Int. Cl. C12Q 1/68; A61K 31/43; A61K 31/545; A61K 38/17

(52) U.S. Cl. 435/6; 514/12; 514/193; 514/200

(57) ABSTRACT

A nucleic acid sequence which regulates the autolytic activity of bacteria is provided. Also provided are polypeptides encoded by these nucleic acid sequences or mutants thereof as well as vector and host cells for expressing these polypeptides. Methods for identifying and using agents which prevent or reduce multiple antibiotic resistance also are provided.

This application is a continuation-in-part of U.S. patent application Ser. No. 10/092,264, filed Mar. 6, 2002 which is incorporated herein by reference and which claims benefit under 35 U.S.C. §119 of U.S. provisional applications Serial Nos. 60/273,791, filed on Mar. 6, 2001; 60/312,546, filed on Aug. 15, 2001; and 60/329,140, filed on Oct. 12, 2001, whose contents are incorporated herein by reference in their entireties.

This invention was made in the course of research sponsored by the National Institutes of Health (NIH Grant No. RO1-AI37142). The U.S. government may have certain rights in this invention.

### BACKGROUND

Staphylococci are hardy and ubiquitous colonizers of human skin and mucous membranes and were among the first human pathogens identified. These bacteria constitute a medically important genera of microbes as they are known to produce two types of disease, invasive and toxigenic.

Invasive infections are characterized generally by abscess formation affecting both skin surfaces and deep tissues. In addition, Staphylococcus aureus (S. aureus) is the second leading cause of bacteremia in cancer patients. Osteomyelitis, septic arthritis, septic thrombophlebitis and acute bacterial endocarditis are also relatively common.

There are also at least three clinical conditions resulting from the toxigenic properties of Staphylococci. The manifestation of these diseases result from the actions of exotoxins as opposed to tissue invasion and bacteremia. These conditions include: Staphylococcal food poisoning, scalded skin syndrome and toxic shock syndrome.

S. aureus are non-motile, non-sporulating gram-positive cocci 0.5-1.5 μm in diameter, that occur singly and in pairs, short chains, and irregular three-dimensional clusters. S. aureus can grow over a wide range of environmental conditions, but they grow best at temperatures between 30° C. and 37° C. and at a neutral pH. They are resistant to desiccation and to chemical disinfection, and they tolerate NaCl concentrations up to 12%. However, growth of S. aureus becomes unusually sensitive to a high-NaCl concentration by decreasing the Ca²⁺ concentration in growth media allowing for autolysis (Ishikawa, Microbiology and Immunology, 2000: 44(2):97-104).

Humans constitute the major reservoir of the S. aureus bacteria. The cross-sectional carriage rate in adults is 15 to 40 percent. The mucous membranes of the anterior nasopharynx are the principal site of carriage. Other sites include the axillae, the vagina, the perineum and occasionally the gastrointestinal tract. Colonization by S. aureus may be intermittent or persistent and is probably influenced by both microbial and host factors as well as by the nature of the competing non-Staphylococcal flora.

The frequency of S. aureus infections has risen dramatically in the past 20 years. This has been attributed to two main factors. The first factor is an increasing population of people with weakened immune systems. The second factor has been the emergence of multiple antibiotic resistant strains. It is no longer uncommon to isolate S. aureus strains which are resistant to some or all of the standard antibiotics. Active efflux of various toxic compounds from the cell to the outer medium is a universal mechanism that bacteria have evolved to protect themselves against the adverse effects of their environments. Antibiotics are expelled from the cells by membrane transporter proteins called multidrug resistance (MDRs) pumps. The NorA protein of S. aureus is an MDR pump that mediates the active efflux of hydrophilic fluoroquinolones from the cell (Ubukata, et al. Antimicrob. Agents Chemother., 1989: 33(9):1535-9), conferring low-level resistance upon the organism. NorA is also capable of transporting additional structurally diverse compounds, indicating that it has a broad substrate specificity. NorR, a protein that shares 60% similarity with B. subtilis MarR and S. aureus SarA regulatory proteins, may function to repress the transcription of NorA (Truong-Bolduc, et al., ASM 2001 General Meeting, May 22, 2001: Abstract A-56).

The rise in the frequency of S. aureus infections has created a demand for both new anti-microbial agents and diagnostic tests for this organism. Accordingly, there is a need for better understanding of factors which regulate infectivity and growth of S. aureus. Genes identified as involved in the infectivity and/or growth of S. aureus include the arlS regulator, involved in adhesion (Fournier and Hooper, Journal of Bacteriology, 2000: 182(14):3955-64), the pbpC gene, which affects the rate of autolysis (Pinho et al., Journal of Bacteriology, 2000: 182(4):1074-9), the anti gene, which encodes a protein having amidase and N-acetylglucosaminidase activity (Sugai, et al., Journal of Bacteriology, 1997:179:2958-2962), lytN (Sugai, Journal of Infection and Chemotherapy, 1997:3:113-127), lytRS (Brunskill and Bayles, Journal of Bacteriology, 1996: 178(19):5810-2), lrgA and lrgB (Fujimoto et al., Journal of Bacteriology, 2000: 182(17):4822-8), and lytM identified in autolysis-deficient mutants of S. aureus (Ramadurai and Jayaswal, Journal of Bacteriology, 1997: 179(11):3625-31).

A new polypeptide of S. aureus which regulates autolytic processes has now been identified. The nucleic acid sequences encoding this polypeptide, referred to herein as Rat, regulator of autolytic activity, which regulates autolytic processes has been cloned and sequenced. Further, it has been shown that mutations in the nucleic acid sequences encoding Rat render S. aureus more susceptible to lysis by antibiotics.

### SUMMARY OF THE INVENTION

The present invention provides a polypeptide of S. aureus and other bacteria that is involved in the control, modulation or regulation (these latter three terms are used as equivalents herein) of autolytic activity and processes in bacteria. The nucleic acid sequence and mutants thereof, as well as the polypeptides encoded thereby are also provided.

An object of the present invention is to provide nucleic acid sequences isolated from S. aureus and other bacteria which regulate autolytic activity in bacteria. The nucleic acid sequences referred to herein are rat (SEQ ID NO: 1), rat mutant (SEQ ID NO: 3), or fragments thereof. In a preferred embodiment, the nucleic acid sequence is rat (SEQ ID NO: 1).

Another object of the present invention is to provide polypeptides (SEQ ID NO: 2) encoded by rat (SEQ ID NO: 1) and vectors and host cells comprising nucleic acid sequences encoding these polypeptides. In a preferred embodiment, the polypeptides have the sequence of SEQ ID NO: 2.

Another object of the present invention is to provide vectors which comprise a transposon element and a nucleic acid sequence which encodes the Rat polypeptide (SEQ ID NO: 2) and host cells comprising this vector.

Another object of the present invention is to provide methods of identifying agents that prevent or reduce multiple antibiotic resistance of S. aureus and other bacteria through interaction with a nucleic acid sequence encoding Rat (SEQ ID NO: 1) or a polypeptide encoded thereby. These agents may be used alone or in combination with an antibiotic such as penicillin to promote lysis of the bacteria.

Accordingly, another object of the present invention is to provide methods for preventing or reducing multiple antibiotic resistance of S. aureus and other bacteria by contacting the bacteria with an agent which interacts with a nucleic acid sequence encoding Rat (SEQ ID NO: 1) or a polypeptide encoded thereby.

Yet another object of the present invention is to provide anti-bacterial agents which comprise a compound which prevents or reduces multiple antibiotic resistance of S. aureus or other bacteria via interaction with a nucleic acid sequence encoding Rat (SEQ ID NO: 1) or a polypeptide encoded thereby.

### BRIEF DESCRIPTION OF THE DRAWINGS

FIG. 1 graphically demonstrates the effect that subinhibitory concentrations of penicillin have on the rat mutant as compared to wild-type.

FIG. 2 graphically represents the viability of the rat mutant as compared to wild-type by propidium iodide staining.

FIG. 3 shows a multiple sequence alignment of Rat with other bacterial transcriptional regulators generated by DIALIGN 2 (Morgenstern, Bioinformatics, 1999: 15:211-218). Staphylococcus aureus Rat (Sa-Rat; SEQ ID NO: 2); Bacillus anthracis MarR (Ba-MarR; SEQ ID NO: 5), accession number NP_{—}658498; Clostridium acetobutylicum MarR (Ca-MarR; SEQ ID NO: 6), accession number NP_{—}350250; Xanthomonas axonopodis MarR (Xa-MarR; SEQ ID NO: 7), accession number NP_{—}641789; Xanthomonas campestris OhrR (Xc-OhrR; SEQ ID NO: 8), accession number AAK62673; and Staphylococcus aureus SarA (Sa-SarA; SEQ ID NO: 9), accession number BAB94445. Capital letters denote the majority. The number of ‘*’ characters below the alignment reflects the degree of conserved residues among sequences.

FIG. 4 shows expression of norA-GFP in wild-type (6390), rat transposon mutant (2529), rat deletion mutant (2530), and sarA mutant (2057) strains. Expression is represented as fluorescence per OD unit and was determined for each strain in early stationary phase.

FIG. 5 shows expression of norA-GFP in wild-type MRSA (COL), COL-rat transposon mutant, and COL-rat deletion mutant strains. Expression is represented as fluorescence per OD unit and was determined for each strain in early stationary phase.

### DETAILED DESCRIPTION OF THE INVENTION

S. aureus is the most prevalent human pathogen in the Staphylococcal genus. It remains a major public health concern due to its tenacity, potential destructiveness and increasing resistance to antimicrobial agents. Much research has been focused on identifying genes or gene products of S. aureus which serve as targets in the development of new antibacterial agents.

Using transposon mutagenesis, a nucleic acid (SEQ ID NO: 1) encoding Rat (regulator of autolytic activity)(SEQ ID NO: 2), which regulates expression of polypeptides involved in autolytic processes of S. aureus, has been identified. The phrase, “which regulates expression of polypeptides involved in autolytic processes” used herein means that the nucleic acid sequence or polypeptide encoded thereby controls, modulates or regulates the expression of polypeptides involved in autolytic processes such as autolytic enzymes (e.g., murein hydrolase, cell wall hydrolase, glycylglycine endopeptidase), polypeptides involved in environmental signaling, antibiotic efflux, the secretion of autolysins, or other autolytic processes. A Tn551 transposon library of S. aureus strain RN6390 was constructed. The library was screened for genes that affected expression of genes encoding the capsular polysaccharide (cap genes—16 genes encoded within the cap operon) of S. aureus. Using the cap promoter linked to the GFP reporter gene (green fluorescent protein), a mutant (ACL2011) was identified that displayed significantly lower cap promoter activity. However, upon growing this mutant, it was discovered that this strain grew poorly in 03GL medium, reaching a maximum optical density of 0.8 when the parental strain could achieve an OD650 nm of 1.3 or higher. The defect was linked to the transposon insertion as this phenotype could be back-crossed into the parental strain yielding strain ACL2529. The region of the mutant chromosome where the transposon was inserted was subsequently sequenced; the nucleic acid sequence flanking the site of the transposon insertion is SEQ ID NO: 3. This nucleic acid sequence was designated rat, or regulator of autolytic activity. The nucleic acid sequence of the rat mutant encodes a truncated protein of 134 residues in length. The rat mutant is a transposon mutant in which a Tn551 transposon is inserted at the 3′ end of the nucleic acid sequence, yielding a truncated protein or polypeptide missing the last 13 amino acid residues of the wild-type protein. It is believed that the rat mutant is a partial knock-out. By “knock-out” it is meant that an alteration in the target gene sequence occurs which results in an alteration of normal function of the target gene product.

Zymogram analysis revealed that the rat mutant strain displays significantly enhanced autolytic activity as compared to the wild-type. This defect in autolytic activity was restored upon complementation of the mutant. The rat mutant was complemented with an integration vector containing the entire rat locus. The complemented strain (ALC2012) grew to a higher optical density (OD650 nm) than the rat mutant strain and had lower cell wall hydrolase activities than the rat mutant.

The native rat sequence (SEQ ID NO: 1) encodes a 17-kDa protein of 147 residues in length. Forty-seven of the 147 residues (32%) are charged and the pI of Rat is predicted to be 7.38.

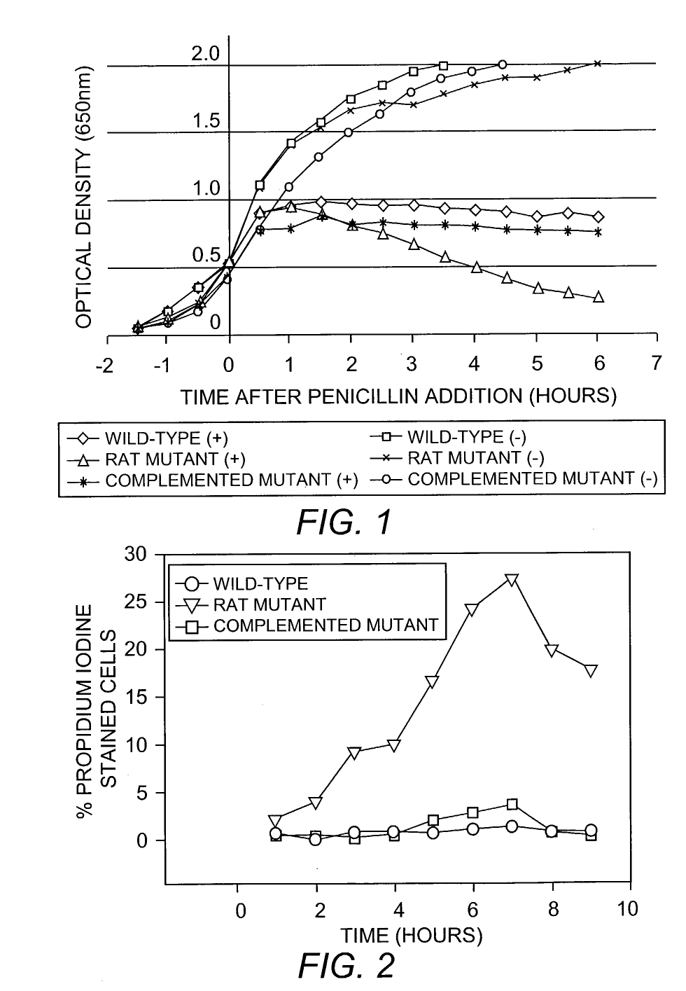

Rat plays a role in regulating autolytic activity of S. aureus. More specifically, in the presence of penicillin, the rat mutant was shown to readily increase lysis as compared to wild-type S. aureus. Furthermore, inactivation of the rat locus renders the S. aureus bacteria sensitive to lysis upon growth beyond the mid-log phase. To evaluate whether the cell lysis of the rat mutant was additive to the effect of a subinhibitory concentration of penicillin, 200 ng/ml of penicillin was added to a growing culture of the rat mutant at an OD650 nm of 0.5, corresponding to the mid-log phase. Upon addition of penicillin, the rat mutant exhibited a further reduction in optical density as the growth cycle progressed (FIG. 1) in contrast to the wild-type strain that displayed no increase in optical density (i.e., no growth). This finding is consistent with the additive effect of penicillin upon the lytic propensity of the rat mutant late in the growth cycle. Similar results were obtained using gentamicin and cephalothin. Upon plating these cultures on agar plates without antibiotic selection, it was found that the rat mutant has 1-2 log more kill than the parental strain. In comparison to the parental strain, the rat mutant with subinhibitory concentrations of penicillin has a 3-4 log kill more than the parental strain with antibiotics.

Zymographic analysis of cell-associated murein hydrolases was performed to analyze the autolytic activity of the rat mutant. Bacterial cells were centrifuged, washed, and resuspended in SDS-gel loading buffer, heated for 3 minutes at 100° C., recentrifuged, and the supernatant applied to a SDS-gel containing heat-killed S. aureus RN4220. Following electrophoresis, the gel was soaked in 0.1 percent TRITON® X-100 at 37° C. overnight to hydrolyze RN4220 cells that had been attacked by autolytic enzymes in the cell extracts. After incubation, the gel was stained with one percent methylene blue and destained in water. Clear bands, indicating zones of murein hydrolase activity, were found to be enhanced in the rat mutant as compared to the wild-type control. As a positive control, a sarA mutant was utilized. The sarA gene normally represses murein hydrolase activity.

The viability of rat mutant cells late in the growth cycle was determined by staining bacterial cells, obtained from different parts of the growth cycle, with propidium iodide. Penetration of the cell with propidium iodide indicates cell death or necrosis. Many of the rat mutant cells picked up the propidium iodide stain as the growth cycle lengthened (FIG. 2), thus accounting for the decrease in optical densities in the rat mutant during the late log phase.

The cell wall morphology of the rat mutant strain differs from the wild-type strain rat mutant cells, undergoing division, exhibited a thicker cell wall than the wild-type strain. The outer contour of the rat mutant was rough while the surface of the wild-type cells was smooth. Irregularities in the outer cell wall, similar to that seen in the rat mutant, have previously been associated with altered autolytic activities in mutants. Bacterium possessing nucleotide sequences with a truncation of the nucleic acid sequences encoding Rat or homolog thereof will also exhibit an increased sensitivity to lysis.

The bacterial cell wall is maintained by competing enzymes involved in the synthesis and lysis of the cell wall. Examples of autolytic enzymes include glucosamidase, muramidase, amidase, and endopeptidase. The synthesis of bacterial cell walls is a dynamic process requiring the precise regulation of both synthetic and autolytic activities. The autolytic activity of many bacteria is carefully controlled during the growth cycle in particular by regulatory elements. A disruption of these regulatory elements alters autolytic activity and leads to premature cell lysis during growth. The rat mutant has a defect in autolytic activity which prevents the mutant from reaching the stationary phase of growth. By northern blot analysis, it was shown that the rat mutation affects the expression of autolytic enyzmes such as LytN, LytM, and Atl. The cell wall hydrolase, lytN, and the glycylglycine endopeptidase, lytM, are up-regulated by the rat mutation. Conversely, the rat mutation down-regulated the regulators of autolytic activity, namely, LytS, LytR, LrgA, LrgB, ArlR, and ArlS. Furthermore, the rat mutation affects the expression of certain other S. aureus genes, e.g., hla, spa, abcA, scdA, and sspA. Both hla and scdA expression are down-regulated by the rat mutation, whereas both spa and abcA expression are up-regulated by the rat mutation.

A rat deletion mutant was also generated. Using a standard allelic replacement approach, the nucleic acid sequences encoding Rat were replaced by an ermC cassette in a double-crossover event with the temperature-sensitive plasmid pCL52.2. With serial and frequent temperature shifts to the non-permissive temperature, a rat deletion mutant (ACL2530) was obtained and confirmed by Southern blot and PCR analyses. No detectable rat mRNA was observed by northern blot analysis in the rat deletion mutant. The rat deletion mutant was complemented with an integration vector containing the entire rat locus and rat expression in this strain (ACL2531) was restored to near normal levels.

Similar to the rat transposon mutant, the rat deletion mutant was unable to grow to an OD650 nm of 1.7 (early-stationary). Furthermore, in the presence of 100 μg penicillin (3×MIC), the rat deletion mutant readily lysed whereas the complemented strain (ACL2531) behaved like wild-type.

TRITON® X-100 sensitivity was determined for rat, sarA, and rat-sarA double-mutant strains. Both rat and sarA mutants displayed sensitivity to TRITON® X-100 concentrations as low as 0.01%. The double-mutant, however, displayed a more severe defect in TRITON® X-100-induced cell lysis.

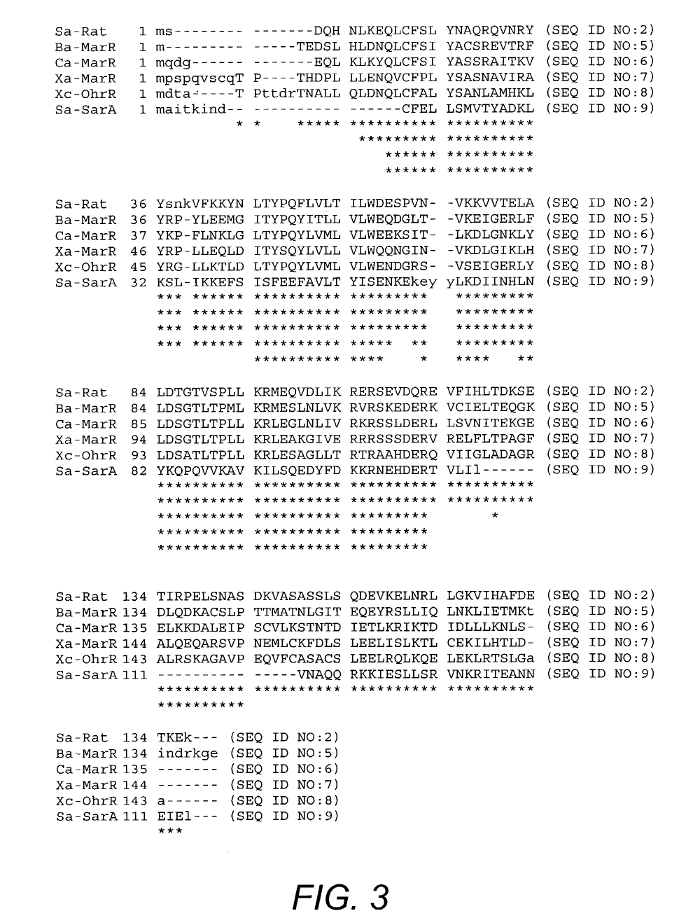

In a comparison of the deduced Rat polypeptide sequence with polypeptides of other microbes, significant sequence similarity was found between Rat and polypeptides that regulate the expression of multiple antibiotic resistance (MAR) genes in both gram positive and gram negative bacteria (FIG. 3). For example, the Rat polypeptide sequence shares 63%, 61% and 61% sequence similarity with Clostridium acetobutylicum, Bacillus anthracis, and Xanthomonas axonopodis MarR polypeptides, respectively.

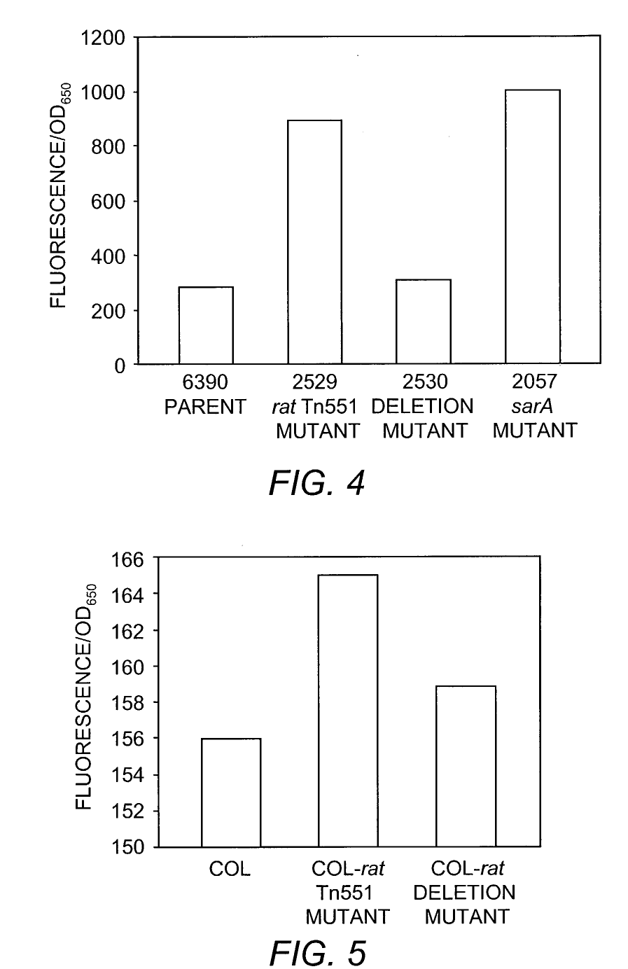

In addition to regulating the expression of autolytic enzyme expression, Rat regulates the efflux of antibiotics. The promoter of norA was fused to GFP to demonstrate that Rat polypeptide regulates the expression of norA. The promoter of the norA gene, corresponding to nucleotides 1-471 of accession number D90119, was PCR-amplified using genomic DNA isolated from strain RN6390 as a template. The amplicon was cloned into the KpnI and XbaI restriction sites of pALC1484, a pSK236-based plasmid harboring GFPᵤᵥᵣ (Cheung, et al., Infection and Immunity, 1998: 66(12):5988-5993). The resulting norA-GFP expression construct was transformed into wild-type S. aureus strain RN6390, the rat Tn551 transposon mutant of RN6390 (ACL2529), the rat deletion mutant of RN6390 (ACL2530) and a sarA mutant strain as a positive control. GFP fluorescence in the rat transposon mutant and sarA mutant strain was approximately four-fold higher than that in either the wild-type or deletion mutant strain (FIG. 4). Similarly, when the norA-GFP expression construct was transformed into wild-type Staphylococcus aureus MRSA strain COL, a methicillin-resistant strain (de Lencastre and Tomasz, Antimicrobial Agents and Chemotherapy, 1994: 38(11): 2590-2598); a rat Tn551 transposon mutant of COL; and a rat deletion mutant of COL, an increase in GFP fluorescence was observed in the rat transposon mutant (FIG. 5). In general, these results demonstrate that a strain deficient in functional Rat polypeptide activity has an increased level of norA gene expression. Therefore, Rat is a negative regulator of norA gene expression.

In a comparison of the nucleic acid sequences encoding Rat with the genomes of other microbes, homologs with significant sequence similarity were identified. Rat or a homolog thereof performs a role in regulating the autolytic activity of bacteria, including but not limited to: Staphylococcus aureus (such as Staphylococcus aureus N315, Staphylococcus aureus strain Mu50, Staphylococcus aureus MSSA strain NCTC 8325, Staphylococcus aureus MRSA strain COL, Staphylococcus epidermidis, and Staphylococcus sciuri), Sinorhizobium species (e.g., meliloti), Listeria species (e.g., monocytogenes), Clostridium species (e.g., acetabutylicum, difficile), Vibrio species (e.g., cholerae), Corynebacterium species (e.g., diptheriae), Brucella species (e.g., suis), Pseudomonas species (e.g., aeruginosa, syringae, putida), Shewanella species (e.g., putrefasciens), Mesorhizobium species (e.g., loti), Caulobacter species (e.g., crescentus), Lactococcus species (e.g., lactis), Mycobacterium species (e.g., smegmatis, leprae, tuberculosis), Burkholderia species (e.g., mallei, pseudomallei), Geobacter species (e.g., sulfurreducens), Treponema species (e.g., denticola), Bacillus species (e.g., stearothermophilus, anthracis, subtilis, halodurnas), Escherichia species (e.g., coli), Enterococcus species (e.g., faecalis), Salmonella species (e.g., dublin, enteriditis, paratyphi, typhi), Klebsiella species (e.g., pneumoniae), Bordetella species (e.g., parapertussis), Actinobacillus species (e.g., actinomycetemcomitans), Streptomyces species (e.g., coelicolor), Streptococcus species (e.g., pyogenes, pneumoniae), Yersinia species (e.g., pestis), Agrobacterium species (e.g., tumefaciens) and Acinetobacter species.

Useful homologous sequences are those which encode a polypeptide which increases bacterial susceptibility to autolysis, increases lysis due to antibiotic administration or regulates the expression of nucleic acid sequences encoding MAR-associated polypeptides such as norA. In a preferred embodiment the polynucleotide sequence is at least 60 percent homologous to the SEQ ID NO: 1 or SEQ ID NO: 3. In a more preferred embodiment the polynucleotide sequence is at least 80 percent homologous to the SEQ ID NO: 1 or SEQ ID NO: 3.

The present invention includes the nucleic acid sequences encoding Rat and a mutant of Rat and polypeptides encoded thereby. For purposes of the present invention, polypeptides encoded by rat and rat mutant are referred to herein as Rat polypeptides and Rat mutant polypeptides, respectively. Exemplary nucleic acid sequences of the present invention are SEQ ID NO: 1 and SEQ ID NO: 3. However, by the term “nucleic acid sequence” it is meant to include any form of DNA or RNA such as cDNA or genomic DNA or mRNA, respectively, encoding a Rat polypeptide or Rat mutant polypeptide, or an active fragment thereof which are obtained by cloning or produced synthetically by well-known chemical techniques. DNA may be double- or single-stranded. Single-stranded DNA may comprise the coding or sense strand or the non-coding or antisense strand. Thus, the term nucleic acid sequence also includes sequences which hybridize under stringent conditions to the above-described polynucleotides. As used herein, the term “stringent conditions” means at least 80% homology at hybridization conditions of 60° C. at 2×SSC buffer.

In a preferred embodiment, the nucleic acid sequence comprises the cDNA of SEQ ID NO: 1 or a homologous sequence or fragment thereof which encodes a polypeptide having similar activity to the polypeptide (SEQ ID NO: 2) encoded by rat. In another preferred embodiment, the nucleic acid sequence comprises the cDNA of SEQ ID NO: 3 or a homologous sequence or fragment thereof which encodes a polypeptide having similar activity to the polypeptide (SEQ ID NO: 4) encoded by the rat mutant. Due to the degeneracy of the genetic code, nucleic acid sequences of the present invention also may comprise other nucleic acid sequences encoding the Rat polypeptide or Rat mutant polypeptide and derivatives, variants or active fragments thereof. The present invention also relates to variants of these nucleic acid sequences which may be naturally occurring, i.e., allelic variants, or mutants prepared by well-known mutagenesis techniques.

The present invention also relates to a conditional mutant whereby the nucleic acid sequence encoding Rat or Rat mutant polypeptides may be expressed under an inducible promoter.

The present invention also relates to vectors comprising nucleic acid sequences of the present invention and host cells which are genetically engineered with these vectors to produce active Rat polypeptides or Rat mutant polypeptides, or fragments thereof. Generally, any vector suitable to maintain, propagate or express the nucleic acid sequences of this invention in a host cell may be used for expression in this regard.

The nucleic acid sequences and polypeptides of the present invention, as well as vectors and host cells expressing the polypeptides are useful as research tools to enhance the understanding of the autolytic process of S. aureus and other bacteria. The methods and compositions of the present invention are believed to be effective in other bacteria having significant homology with the polynucleotide sequence of rat or the rat mutant.

Further, the Rat compositions are useful in the identification of agents which interact with the Rat polypeptide or the nucleic acid sequence encoding Rat to modulate autolytic activity of the bacteria. By “interact” it is meant that the agent increases or decreases expression of products of nucleic acid sequences encoding Rat or increases or decreases activity of a Rat polypeptide. A product of a nucleic acid sequence encoding Rat includes mRNA and polypeptides. Assays for identifying such agents are well-known in the art and may be conducted in vivo or in vitro. For example, isolated Rat polypeptides or test cells which contain nucleic acid sequences encoding Rat polypeptides are contacted with test agents and the interaction of the agent with the Rat polypeptide or the nucleic acid sequence encoding Rat is determined. General approaches which may be used to determine the interaction between the agent and the Rat polypeptide or nucleic acid sequence encoding Rat include, but are not limited to, competitive binding assays between the agent and an antibody specific for Rat polypeptide or DNA-DNA or DNA-RNA hybridization assays to assess the level of expression of products of the nucleic acid sequences encoding Rat.

In a preferred embodiment, agents will decrease, interfere with or inhibit expression of products of nucleic acid sequences encoding Rat so that the bacteria is lysed more easily. Examples of such agents include, but are not limited to, antisense molecules, RNAi or ribozymes targeted to nucleic acid sequence encoding Rat which inhibit the expression of products of nucleic acid sequences encoding Rat, means for introduction of mutations into nucleic acid sequence encoding Rat which inhibit expression of products of nucleic acid sequences encoding Rat or produce a Rat polypeptide with decreased activity, and small organic molecules or peptides which are capable of inhibiting activity of the Rat polypeptides or the nucleic acid sequences encoding Rat themselves (e.g., by binding to the promoter region of the gene to inhibit transcription and subsequent expression). The active site of the Rat polypeptide, could be used to simulate activity in the mutant. Alternatively, a small compound library may be used to screen for agents which augment the lytic activity of nucleic acid sequences encoding Rat or Rat mutant polypeptides. This augmentation of lytic activity may be monitored by binding of the small compound to the Rat polypeptide or Rat mutant polypeptide and determining the ability of the small compound to induce cell lysis.

In another preferred embodiment, agents will increase, activate or stimulate expression of products of nucleic acid sequences encoding Rat or increase, activate or stimulate the activity of a Rat polypeptide to repress, decrease, or inhibit the expression of products of nucleic acid sequences encoding multiple antibiotic resistance polypeptides so that the efflux of antibiotics is reduced or prevented. Examples of such agents include, but are not limited to, means for introduction of mutations into the nucleic acid sequences encoding Rat which stimulate expression of products of nucleic acid sequences encoding Rat or produce a Rat polypeptide with increased activity and small organic molecules or peptides which are capable of increasing or stimulating activity of the Rat polypeptides or the nucleic acid sequences encoding Rat themselves (e.g., by binding to the promoter region of the gene to promote transcription and subsequent expression).

Accordingly, one aspect of the present invention provides an analog library to produce a very large number of potential molecules or agents for regulating the expression of products of nucleic acid sequences encoding Rat, and in general the greater the number of analogs in the library, the greater the likelihood that at least one member of the library will effectively regulate the expression of products of nucleic acid sequences encoding Rat. Designed libraries following a particular template structure and limiting amino acid variation at particular positions are much preferred, since a single library may encompass all the designed analogs and the included sequences will be known and presented in roughly equal numbers. By contrast, random substitution at only six positions in an amino acid sequence provides over 60 million analogs, which is a library size that begins to present practical limitations even when utilizing screening techniques as powerful as phage display. Libraries larger than this would pose problems in handling, e.g., fermentation vessels would need to be of extraordinary size, and more importantly, the likelihood of having all of the planned polypeptide sequence variations represented in the prepared library would decrease sharply. It is therefore preferred to create a designed or biased library, in which the amino acid positions designated for variation are considered so as to maximize the effect of substitution on the Rat regulation characteristics of the analog, and the amino acid residues allowed or planned for use in substitutions are limited.

The use of replicable genetic packages, such as the bacteriophages, is one method of generating novel polypeptide entities that regulate the expression of products of nucleic acid sequences encoding Rat. This method generally consists of introducing a novel, exogenous DNA segment into the genome of a bacteriophage (or other amplifiable genetic package) so that the polypeptide encoded by the non-native DNA appears on the surface of the phage. When the inserted DNA contains sequence diversity, then each recipient phage displays one variant of the template amino acid sequence encoded by the DNA, and the phage population (library) displays a vast number of different but related amino acid sequences.

Such techniques make it possible not only to screen a large number of potential binding molecules but make it practical to repeat the binding/elution cycles and to build secondary, biased libraries for screening analog-displaying packages that meet the initial criteria.

It is well-known to those skilled in the art that it is possible to replace peptides with peptidomimetics. Peptidomimetics are generally preferable as therapeutic agents to peptides owing to their enhanced bioavailability and relative lack of attack from proteolytic enzymes. Accordingly, the present invention also provides peptidomimetics and other lead compounds which may be identified based on data obtained from structural analysis of Rat. A potential analog may be examined through the use of computer modeling using a docking program such as GRAM, DOCK, or AUTODOCK. This procedure may include computer fitting of potential analogs. Computer programs also may be employed to estimate the attraction, repulsion, and steric hindrance of an analog to a potential binding site. Generally the tighter the fit (e.g., the lower the steric hindrance, and/or the greater the attractive force) the more potent the potential analog will be since these properties are consistent with a tighter binding constant. Furthermore, the more specificity in the design of a potential analog the more likely that the analog will not interfere with other properties of the expression of Rat. This will minimize potential side-effects due to unwanted interactions with other proteins.

Initially a potential analog could be obtained by screening a random peptide library produced by a recombinant bacteriophage, for example, or a chemical library. An analog ligand selected in this manner could then be systematically modified by computer modeling programs until one or more promising potential ligands are identified.

Such computer modeling allows the selection of a finite number of rational chemical modifications, as opposed to the countless number of essentially random chemical modifications that could be made, and of which any one might lead to a useful agent. Thus, the three-dimensional structure and computer modeling, provides that a large number of agents may be rapidly screened and a few likely candidates may be determined without the laborious synthesis of untold numbers of agents.

Once a potential peptidomimetic or lead compound is identified it may be either selected from a library of chemicals commercially available from most large chemical companies including Merck, GlaxoWelcome, Bristol Meyers Squibb, Monsanto/Searle, Eli Lilly, Novartis and Pharmacia UpJohn, or alternatively the potential ligand is synthesized de novo. As mentioned above, the de novo synthesis of one or even a relatively small group of specific compounds is reasonable in the art of designing compounds.

Agents of the present invention may comprise antibodies against the Rat polypeptide. Antibodies against the Rat polypeptide may facilitate selective delivery of a cytotoxic agent to S. aureus or other bacteria. Alternatively, antibodies may serve as the agent, binding to the Rat polypeptide thereby inhibiting activity. The Rat polypeptides or epitope bearing fragments thereof may be used as immunogens to produce antibodies immunospecific for such polypeptides. Various techniques well-known in the art may be used routinely to produce antibodies (Kohler, G. and Milstein, C., Nature 1975: 256: 495-497; Kozbor et al., Immunology Today, 1983: 4: 72; Cole et al., Monoclonal Antibodies and Cancer Therapy, 1985: pp 77-96).

Accordingly, the present invention also provides agents identified as inhibitors of expression of products of nucleic acid sequences encoding Rat or Rat polypeptide activity and methods for using these agents to increase lysis of S. aureus and other bacteria, thereby inhibiting their growth and infectivity. Furthermore, the invention provides agents identified as stimulators or activators of expression of products of nucleic acid sequences encoding Rat or Rat polypeptide activity and methods for using these agents to decrease, repress, or inhibit multiple antibiotic resistance gene expression of S. aureus and other bacteria, thereby preventing or reducing bacterial resistance to antimicrobial agents. These agents may be incorporated into a pharmaceutical composition and administered to a host to inhibit growth and infectivity of S. aureus and other bacteria in the host. The term “host” as used herein includes humans.

Pharmaceutical compositions of the present invention comprise an effective amount of an agent which alters the expression of a product of a nucleic acid sequence encoding Rat or an activity of the Rat polypeptide and a pharmaceutically acceptable vehicle. Such pharmaceutical compositions may be prepared by methods and contain vehicles which are well-known in the art. A generally recognized compendium of such methods and ingredients is Remington's Pharmaceutical Sciences (A. R. Gennaro ed. 1985. Mack Publishing Co.). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

By “effective amount” it is meant an amount which inactivates the expression or the activity of a product of a nucleic acid sequence encoding Rat and renders S. aureus or other bacteria susceptible to killing through cell lysis. Alternatively, an effective amount may be an amount which activates the expression or the activity of a product of a nucleic acid sequence encoding Rat and renders S. aureus or other bacteria susceptible to antibiotic killing through prevention or reduction of multidrug resistance transporter protein expression. The pharmaceutical compositions may be administered to a host, preferably a human, to inhibit the growth of S. aureus or other bacteria in the host.

The pharmaceutical composition may be administered alone, or in combination with an antibiotic such as a penicillin (e.g., penicillin, ampicillin, carbenicillin, methicillin, oxacillin), a penam (e.g., imipenem), a cephalosporin (e.g., cephalothin, cefoxitin, cefotaxime), an aminoglycoside (e.g., gentamicin, kanamycin, tobramycin, amikacin, streptomycin, neomycin), a tetracycline (e.g., tetracycline, doxycycline), a macrolide (e.g., erythromycin, clarithromycin, azithromycin), a quinolone (e.g., ciprofloxacin, gatifloxacin, levofloxacin), rifampin or vancomycin to enhance killing or lysis of the bacteria. Furthermore, the pharmaceutical compositions may be administered in combination with known multidrug resistance pump inhibitors such as 5′-methoxyhydnocarpin-D and pheophorbide A. Pharmaceutical compositions of the present invention may be administered by various routes, including, but not limited to, topically, intramuscularly, intraperitoneally, intranasally, orally, subcutaneously, or intravenously.

The embodiments herein described are not meant to be limiting to the invention. Those of skill in the art will appreciate the invention may be practiced by using numerous chemical entities and by numerous methods all within the breadth of the following claims.

### EXAMPLE 1

Bacterial Strains, Plasmids and Growth Conditions

Phage φ11 and 80α were used as the transducing phage for S. aureus strains. S. aureus cells were grown at 37° C. with aeration in CYGP or 03GL broth or Tryptic soy broth (TSB) supplemented with antibiotics when necessary. Luria-Bertani (LB) broth was used for cultivating E. coli

Antibiotics were used at the following concentrations for S. aureus: erythromycin, 5 μg/ml; tetracycline, 3 μg/ml; chloramphenicol, 10 μg/ml; kanamycin, 50 μg/ml; for E. coli ampicillin, 50 μg/ml; spectinomycin, 75 μg/ml.

### EXAMPLE 2

Genetic Manipulations in E. coli and S. aureus

Recombinant plasmid construction was performed in E. coli DH5α™. Standard molecular biology and recombinant DNA techniques were used (Sambrook et al. (1989) Molecular Cloning, a Laboratory Manual, Cold Spring Harbor Laboratories, New York).

S. aureus strain RN4220, a restriction deficient derivative of strain 8325-4, was used as the initial recipient for the transformation of plasmid constructs, using well-known electroporation methods.

A Tn551 transposon library was constructed in RN6390 (Cheung, et al., Journal of Bacteriology, 1995: 177(11):3220-6), using the temperature-sensitive plasmid pI258 as the delivery vehicle for the transposon. A reporter plasmid containing the cap5 promoter driving the promoterless gfpᵤᵥᵣ was electroporated into the transposon library to select for mutants that had decreased GFP fluorescence. One mutant, ALC2011, had a defect in autolytic activities.

To construct a rat deletion mutant, upstream and downstream sequences flanking the Rat open reading frame were PCR-amplified, using RN6390 chromosomal DNA as a template. PCR primers used for amplification of the upstream fragment were 5′-CGA GAG CTC TAA ATG ACA CAT AAC CTT TCA-3′ (SEQ ID NO: 10) and 5′-TCC CCC GGG ATT GGT AAT CAT TAA AAA GTT-3′ (SEQ ID NO: 11), and for downstream fragment 5′-CCG GTC GAC CTT GAT TAG CTA GTA ATT GTT-3′ (SEQ ID NO: 12) and 5′-AAC TGC AGC GCT AGT TAC AGT CAT AGT TT-3′ (SEQ ID NO: 13). The upstream fragment was cloned into Smal-Sacd and the downstream into SalI-PstI sites of the temperature-sensitive shuttle vector pCL52.2. The antibiotic marker ermC (1.2-kb fragment) was inserted between these two fragments such that the ermC gene was divergent from rat. This recombinant plasmid (pALC2463) was used to transform RN4220. Transformants were selected for erythromycin resistant colonies at 30° C. The recombinant plasmid was isolated from RN4220 and introduced into RN6390 by electroporation. S. aureus strain RN6390, harboring plasmid pALC2463, was grown overnight at 30° C. in TSB to allow for plasmid replication and propagated at 42° C., a non-permissive temperature for replication of pCL52.2. This cycle was repeated 4-5 times and cells were plated on 03GL plates containing erythromycin or erythromycin and tetracycline to select for tetracycline sensitive and erythromycin resistant colonies, representing mutants with double-crossovers. After six cycles, putative mutants were obtained. PCR and Southern blot analysis confirmed the double-crossover events. One clone, designated ALC2530, was further analyzed.

To complement the rat mutation, a 3.2-kb rat fragment was amplified from RN6390 DNA with primers 5′-CGA GAG CTC TAA ATG ACA CAT AAC CTT TCA-3′ (SEQ ID NO: 14) and 5′-AAC TGC AGC GCT AGT TAC AGT CAT AGT TT-3′ (SEQ ID NO: 15) and cloned into pCL84, a plasmid that integrates into the lipase gene of S. aureus (Lee, et al., Gene, 1991: 103:101-105.), to yield pALC2464. CYL316, an RN4420 derivative, was transformed with pALC2464 and tetracycline resistant colonies were selected. For correct chromosomal integration, colonies were checked for negative lipase activity on egg-yolk agar plates. The integrated plasmid containing the complete rat locus in RN4220 was transduced into the rat transposon (ALC2529) and deletion (ALC2530) mutant strains, using phage φ11. Tetracycline resistance strains lacking lipase activity were analyzed for the complementation phenotype.

To generate other rat transposon and rat deletion mutants, φ11 and 80α phage lysates of ALC2529 and ALC2530 were used to infect COL strains.

### EXAMPLE 3

Zymographic Analysis

Cell-associated murein hydrolase activity was detected using SDS-PAGE zymographic analysis. Briefly, an equal number of cells from each strain were centrifuged, washed and resuspended in SDS-gel loading buffer, heated for three minutes at 100° C., and centrifuged to obtain supernatant. Supernatants were separated on a 12% SDS-polyacrylamide gel containing RN4220 cells (1 mg wet weight per ml, heat-killed). After electrophoresis, gels were washed with water and incubated for 12 hours in 25 mM Tris-HCl (pH 8.0) containing 1% TRITON® X-100 at 37° C. Gels were stained with 1% methylene blue and clear zones of hydrolysis were observed against a dark background.

### EXAMPLE 4

Transmission Electron Microscopy

Bacterial cells in the mid-exponential phase of growth were washed four times with PBS and then fixed with 2% gluteraldehyde/1% paraformaldehyde in 0.1 M sodium cacodylate, pH 7.4, overnight. The cells were washed three times with 0.1 M sodium cacodylate, pH 7.4. The cells were then suspended in 1% OsO₄ in 0.1 M sodium cacodylate, pH 7.4, for one hour at room temperature and subsequently rinsed with 0.1 M sodium cacodylate, pH 7.4, and distilled water. En bloc stain: 1-2% Uranyl Acetateaq for 30 minutes at room temperature, in the dark. The cells were rinsed with water and dehydrated in a series of ethanol washes. Prior to fixing, the cells were washed twice with propylene oxide. The pellet was immersed in LX112:PO (1.5:1). Vials were capped for two hours. Caps were removed and the vials were desiccated overnight under vacuum. Cellular pellets were then transferred to the BEEM capsule and filled with fresh LX112. The solution was centrifuged for 30 minutes at 1,000 rpm to pellet cells to the bottom of the capsule. Cells were desiccated overnight under vacuum and subsequently cut in thin sections and stained with 1% uranyl acetate.

### EXAMPLE 5

Autolytic Assays

The effect of TRITON® X-100 on growing cells was determined by diluting cultures grown overnight to an OD650 nm of 0.1 in TSB with varying concentrations of TRITON® X-100. Cells were incubated at 37° C. with shaking and the optical density was recorded hourly for seven to eight hours.

TRITON® X-100-induced autolysis assays were conducted by diluting cultures grown overnight to an OD650 nm of 0.05 in TSB containing 1 M NaCl. Cells were allowed to grow at 37° C. with shaking until an OD650 nm of 0.7 was reached. Cells were harvested, washed twice with ice cold water and then resuspended in the same volume of 0.05 M Tris-HCl, pH 7.2, containing 0.05% TRITON® X-100. Cells were incubated at 30° C. with shaking and lysis was determined by measuring absorbance at OD600 nm at 30 minutes intervals.

Penicillin sensitivity assays were conducted by diluting rat mutant cultures grown overnight to an OD650 nm of 0.05 in TSB containing the appropriate antibiotics to maintain selection. Cultures were grown at 37° C. with shaking to reach exponential phase (OD650 nm=0.5). Penicillin G was added to a concentration of 0.1 μg/ml. Optical densities at 650 nm were measured every hour for eight to nine hours.

### EXAMPLE 6

RNA Isolation, Northern Blot Analysis and Primer Extension

Overnight cultures of S. aureus were diluted 1:100 in CYGP and grown to mid-log (OD650 nm=0.7), late-log (OD650 nm=1.1), and early stationary (OD650 nm=1.7) phases. The cells were harvested and processed with a TRIZOL™ isolation kit (Gibco BRL, Gaithersburg, Md.) in combination with 0.1-mm-diameter sirconia-silica beads in a reciprocating shaker (Biospec, Inc. Bartlesville, Okla.). Fifteen micrograms of each sample was electrophoresed in a 1.5% agarose-0.66 M formaldehyde gel in morpholinepropanesulfonic acid (MOPS) running buffer (20 mM MOPS, 10 mM sodium acetate, 2 mM EDTA, pH 7.0). Northern blotting of RNA was performed with the TURBOBLOTTER™ alkaline transfer system (Schleicher & Schuell, Keene, N.H.) onto HYBOND™ N⁺ membranes (AMERSHAM™, Arlington Heights, Ill.). For detecting lytSR, lrgAB, arlRS, lytM, lytN, atl, pbp2, pbp4, abcA, sspA, scdA, spa, hla and rat, gel-purified DNA probes were radiolabeled with α-³²P dCTP by a random-primed DNA labeling kit (Roche Diagnostics GmbH, Mannheim, GER) and hybridized under aqueous phase conditions at 65° C. The blots were subsequently washed and exposed to autoradiography film using methods well-known to one of skill in the art.

### EXAMPLE 7

GFPᵤᵥᵣ Reporter Constructs

Regulatory activity of rat was analyzed using norA-GFP fusions. The promoter of norA (nucleotides 1-471 of accession number D90119) was cloned into the shuttle vector pALC1484, which is a derivative of pSK236, containing the promoterless gfpᵤᵥᵣ gene. A transcriptional fusion to the gfpᵤᵥᵣ reporter gene was thus created. Restriction analysis and DNA sequencing confirmed the orientation and authenticity of the promoter fragment. Recombinant plasmids were first introduced into S. aureus strain RN4220 by electroporation. Plasmids purified from RN4220 transformants were then electroporated into RN6390 and isogenic rat mutants.

Overnight cultures of S. aureus strains harboring the recombinant plasmids were diluted 1:100 and grown at 37° C. with shaking in tryptic soy broth containing chloramphenicol (10 μg/ml). Aliquots (200 μl) were transferred hourly to microtiter plates to assay for cell density (OD650 nm) and fluorescence for 10 hour in a FL600 fluorescence reader (BioTek Instrument, Winooski, Vt.). Promoter activation was plotted as mean fluorescence/OD650 nm ratio, using the average values from triplicate readings.

What is claimed is:

1. A method for identifying agents which prevent or reduce multiple antibiotic resistance in a bacterium comprising contacting a nucleic acid sequence encoding Rat (SEQ ID NO: 1) or a product thereof with an agent and determining whether said agent interacts with the nucleic acid sequence encoding Rat or the product thereof wherein such an interaction prevents or reduces multiple antibiotic resistance in the bacterium.

2. The method of claim 1 wherein the bacterium is Staphylococcus aureus.

3. The method of claim 1 wherein the bacterium comprises Staphylococcus, Sinorhizobium, Listeria, Clostridium, Bacillus, Corynebacterium, Brucella, Pseudomonas, Shweanella, Mesorhizobium, Caulobacter, Lactococcus, Mycobacterium, Burkholderia, Geobacter, Treponema, Vibrio, Escherichia, Enterococcus, Salmonella, Klebsiella, Agrobacterium, Yersinia, BordeLella, Actinobacillus, Streptomyces, Streptococcus, or Acinetobacter.

4. A method of preventing or reducing multiple antibiotic resistance in a bacterium comprising contacting a bacterium with an agent which regulates the expression or activity of a polypeptide encoded by SEQ ID NO: 1.

5. The method of claim 4 further comprising an antibiotic.

6. The method of claim 4 wherein the bacterium is Staphylococcus aureus.

7. The method of claim 4 wherein the bacterium comprises Staphylococcus, Sinorhizobium, Listeria, Clostridium, Bacillus, Corynebacterium, Brucella, Pseudomonas, Shweanella, Mesorhizobium, Caulobacter, Lactococcus, Mycobacterium, Burkholderia, Geobacter, Treponema, Vibrio, Escherichia, Enterococcus, Salmonella, Klebsiella, Agrobacterium, Yersinia, Bordetella, Actinobacillus, Streptomyces, Streptococcus, or Acinetobacter.

8. A pharmaceutical composition for use as an anti-bacterial agent comprising a pharmaceutically acceptable vehicle and either an agent which regulates the expression or activity of a polypeptide encoded by SEQ ID NO: 1.

9. The pharmaceutical composition of claim 8 further comprising an antibiotic.


SEQUENCE LISTING (15 sequences)

SEQ ID NO: 1 - DNA, 444 bp, Staphylococcus aureus
  CDS (1)..(444)
atgtctgatcaacataatttaaaagaacagctatgctttagtttgtacaatgctcaaagacaagttaatcgctactactctaacaaagtttttaagaagtacaatctaacatacccacaatttcttgtcttaacaattttatgggatgaatctcctgtaaacgtcaagaaagtcgtaactgaattagcactcgatactggtacagtatcaccattattaaaacgaatggaacaagtagacttaattaagcgtgaacgttccgaagtcgatcaacgtgaagtatttattcacttgactgacaaaagtgaaactattagaccagaattaagtaatgcatctgacaaagtcgcttcagcttcttctttatcgcaagatgaagttaaagaacttaatcgcttattaggtaaagtcattcatgcatttgatgaaacaaaggaaaaataa

SEQ ID NO: 2 - protein, 147 aa, Staphylococcus aureus
MSDQHNLKEQLCFSLYNAQRQVNRYYSNKVFKKYNLTYPQFLVLTILWDESPVNVKKVVTELALDTGTVSPLLKRMEQVDLIKRERSEVDQREVFIHLTDKSETIRPELSNASDKVASASSLSQDEVKELNRLLGKVIHAFDETKEK

SEQ ID NO: 3 - DNA, 402 bp, Staphylococcus aureus
  CDS (1)..(402)
atgtctgatcaacataatttaaaagaacagctatgctttagtttgtacaatgctcaaagacaagttaatcgctactactctaacaaagtttttaagaagtacaatctaacatacccacaatttcttgtcttaacaattttatgggatgaatctcctgtaaacgtcaagaaagtcgtaactgaattagcactcgatactggtacagtatcaccattattaaaacgaatggaacaagtagacttaattaagcgtgaacgttccgaagtcgatcaacgtgaagtatttattcacttgactgacaaaagtgaaactattagaccagaattaagtaatgcatctgacaaagtcgcttcagcttcttctttatcgcaagatgaagttaaagaacttaatcgcttatta

SEQ ID NO: 4 - protein, 134 aa, Staphylococcus aureus
MSDQHNLKEQLCFSLYNAQRQVNRYYSNKVFKKYNLTYPQFLVLTILWDESPVNVKKVVTELALDTGTVSPLLKRMEQVDLIKRERSEVDQREVFIHLTDKSETIRPELSNASDKVASASSLSQDEVKELNRLL

SEQ ID NO: 5 - protein, 150 aa, Bacillus anthracis
MTEDSLHLDNQLCFSIYACSREVTRFYRPYLEEMGITYPQYITLLVLWEQDGLTVKEIGERLFLDSGTLTPMLKRMESLNLVKRVRSKEDERKVCIELTEQGKDLQDKACSLPTTMATNLGITEQEYRSLLIQLNKLIETMKTINDRKGE

SEQ ID NO: 6 - protein, 143 aa, Clostridium acetobutylicum
MQDGEQLKLKYQLCFSIYASSRAITKVYKPFLNKLGLTYPQYLVMLVLWEEKSITLKDLGNKLYLDSGTLTPLLKRLEGLNLIVRKRSSLDERLLSVNITEKGEELKKDALEIPSCVLKSTNTDIETLKRIKTDIDLLLKNLS

SEQ ID NO: 7 - protein, 152 aa, Xanthomonas axonopodis
MPSPQVSCQTPTHDPLLLENQVCFPLYSASNAVIRAYRPLLEQLDITYSQYLVLLVLWQQNGINVKDLGIKLHLDSGTLTPLLKRLEAKGIVERRRSSSDERVRELFLTPAGFALQEQARSVPNEMLCKFDLSLEELISLKTLCEKILHTLD

SEQ ID NO: 8 - protein, 153 aa, Xanthomonas campestris
MDTATPTTDRTNALLQLDNQLCFALYSANLAMHKLYRGLLKTLDLTYPQYLVMLVLWENDGRSVSEIGERLYLDSATLTPLLKRLESAGLLTRTRAAHDERQVIIGLADAGRALRSKAGAVPEQVFCASACSLEELRQLKQELEKLRTSLGAA

SEQ ID NO: 9 - protein, 124 aa, Staphylococcus aureus
MAITKINDCFELLSMVTYADKLKSLIKKEFSISFEEFAVLTYISENKEKEYYLKDIINHLNYKQPQVVKAVKILSQEDYFDKKRNEHDERTVLILVNAQQRKKIESLLSRVNKRITEANNEIEL

SEQ ID NO: 10 - DNA, 30 bp, Artificial Sequence
  Synthetic oligonucleotide primer
cgagagctctaaatgacacataacctttca

SEQ ID NO: 11 - DNA, 30 bp, Artificial Sequence
  Synthetic oligonucleotide primer
tcccccgggattggtaatcattaaaaagtt

SEQ ID NO: 12 - DNA, 30 bp, Artificial Sequence
  Synthetic oligonucleotide primer
ccggtcgaccttgattagctagtaattgtt

SEQ ID NO: 13 - DNA, 29 bp, Artificial Sequence
  Synthetic oligonucleotide primer
aactgcagcgctagttacagtcatagttt

SEQ ID NO: 14 - DNA, 30 bp, Artificial Sequence; identical to SEQ ID NO: 10
  Synthetic oligonucleotide primer

SEQ ID NO: 15 - DNA, 29 bp, Artificial Sequence
  Synthetic oligonucleotide primer
aactgcagcgctagttacagtcatagttt